(19) United States
(12) Patent Application Publication
(10) Pub. No.: US 2026/0076765 A1
(43) Pub. Date: Mar. 19, 2026

(54) ULTRASOUND-BASED MIXED REALITY ASSISTANCE FOR ORTHOPEDIC SURGERIES

(71) Applicant: Howmedica Osteonics Corp., Mahwah, NJ (US)

(72) Inventor: Julia C. Alspaugh, Memphis, TN (US)

(21) Appl. No.: 19/107,381

(22) PCT Filed: Aug. 29, 2023
(86) PCT No.: PCT/US2023/031380

§ 371 Date: Feb. 27, 2025

Related U.S. Application Data
(60) Provisional application No. 63/373,949, filed on Aug. 30, 2022.

(51) Int. Cl. A61B 90/00; A61B 34/10

(52) U.S. Cl. CPC A61B 90/37; A61B 34/10; A61B 2034/105; A61B 2090/365; A61B 2090/378

(57) ABSTRACT

An example method includes obtaining, by a processing system comprising one or more processors implemented in circuitry, ultrasound imaging data representing a non-viewable portion of a bone of a patient; identifying, by the processing system, based on the ultrasound imaging data, a soft tissue attachment point, wherein the soft tissue attachment point is a point on the non-viewable portion of the bone at which a soft tissue structure is attached to the bone; and causing, by the processing system, a Mixed Reality visualization device to display a virtual indicator superimposed on a viewable portion of the bone at a location corresponding to the soft tissue attachment point.

This application claims the benefit of U.S. Provisional Patent Application 63/373,949, filed Aug. 30, 2022, the entire content of which is incorporated by reference.

### BACKGROUND

Surgical joint repair procedures involve repair and/or replacement of a damaged or diseased joint. Many times, a surgical joint repair procedure, such as joint arthroplasty as an example, involves replacing the damaged joint with a prosthetic that is implanted into the patient's bone. Prior to implanting the prosthetic, the surgical site should be clear of resected bone, soft tissue, and other native anatomy expecting to be extracted to allow for proper placement of the prosthetic joint at the surgical site. To verify the surgical site is clear of resected bone and other debris, the surgeon is limited to a visual inspection and/or repetitive medical imaging of the surgical site.

Today, virtual visualization tools that use three-dimensional modeling of bone shapes to facilitate planning for joint repairs and replacements are available to surgeons. These tools can assist surgeons during the pre-operative, intra-operative, and post-operative stages of the surgical joint repair procedure to improve surgical outcomes by customizing a surgical plan for each patient.

### SUMMARY

This disclosure describes techniques in which mixed reality (MR) guidance and an ultrasound probe are used to help a clinician acquire information regarding the soft tissue structures involved in a surgery, such as an orthopedic surgery. As described here, a computing system may obtain reference data that depicts at least one bone of the patient. Example types of reference data may include one or more computed tomography (CT) images, magnetic resonance imaging (MRI) images, nuclear magnetic resonance (NMR) images, and so on. Additionally, the computing system may use the reference data to generate virtual indicators. The virtual indicators provide location information to a clinician regarding soft tissue structure attached to the bone of the patient based on data collected from an ultrasound probe. The computing system may cause a head-mounted MR visualization device to output the virtual indicators to the clinician.

This disclosure describes techniques for intra-operative surgical planning, intra-operative surgical guidance, and intra-operative surgical tracking using mixed reality. In some examples, the disclosure also describes surgical items and/or methods for performing surgical joint repair procedures.

In some examples, this disclosure describes a method comprising: obtaining, by a processing system comprising one or more processors implemented in circuitry, ultrasound imaging data representing a non-viewable portion of a bone of a patient; identifying, by the processing system, based on the ultrasound imaging data, a soft tissue attachment point, wherein the soft tissue attachment point is a point on the non-viewable portion of the bone at which a soft tissue structure is attached to the bone; and causing, by the processing system, a Mixed Reality (MR) visualization device to display a virtual indicator superimposed on a viewable portion of the bone at a location corresponding to the soft tissue attachment point.

In another example, this disclosure describes a computing system comprising: a storage system configured to store data depicting an ultrasound image representing a non-viewable portion of a bone of a patient; a processing system comprising one or more processors implemented in processing circuitry, the processing system configured to: obtain ultrasound imaging data representing the non-viewable portion of the bone of the patient; identify, based on the ultrasound imaging data, a soft tissue attachment point, wherein the soft tissue attachment point is a point on the non-viewable portion of the bone at which a soft tissue structure is attached to the bone; and cause, a Mixed Reality (MR) visualization device to display a virtual indicator superimposed on a viewable portion of the bone at a location corresponding to the soft tissue attachment point.

In another example, this disclosure describes a non-transitory computer-readable storage medium having instructions stored thereon that, when executed, cause a processing system to perform any of methods of this disclosure.

The details of various examples of the disclosure are set forth in the accompanying drawings and the description below. Various features, objects, and advantages will be apparent from the description, drawings, and claims.

### BRIEF DESCRIPTION OF THE DRAWINGS

FIG. 1 is a conceptual diagram illustrating an example system in which one or more techniques of this disclosure may be performed.

FIG. 2 is a conceptual diagram anterior view of an example left ankle ready for extracting a tibial bone fragment and a talar bone fragment during a total ankle replacement surgery, in accordance with one or more techniques of this disclosure.

FIG. 3 is a conceptual diagram illustrating a Mixed Reality (MR) visualization of virtual indicators in the surgical site, in accordance with one or more techniques of this disclosure.

FIG. 4 is a conceptual diagram illustrating an example computing system in accordance with one or more techniques of this disclosure.

FIG. 5 is a block diagram illustrating example components of a visualization device for use in an MR system, according to an example of this disclosure.

FIG. 6 is a flowchart illustrating an example operation of a system, in accordance with one or more techniques of this disclosure.

FIG. 7 is a conceptual diagram illustrating an example MR visualization in which virtual elements indicate locations of a detachment tool and a resection surface in accordance with one or more techniques of this disclosure.

### DETAILED DESCRIPTION

Certain examples of this disclosure are described with reference to the accompanying drawings, wherein like reference numerals denote like elements. It should be understood, however, that the accompanying drawings illustrate only the various implementations described herein and are not meant to limit the scope of various technologies described herein. The drawings show and describe various examples of this disclosure.

In the following description, numerous details are set forth to provide an understanding of the present invention. However, it will be understood by those skilled in the art that the present invention may be practiced without these details and that numerous variations or modifications from the described examples may be possible.

Orthopedic surgery can involve implanting one or more prosthetic devices to repair or replace a patient's damaged or diseased joint. Today, virtual surgical planning tools are available that use image data of the diseased or damaged joint to generate an accurate three-dimensional bone model that can be viewed and manipulated preoperatively by the surgeon. These tools can enhance surgical outcomes by allowing the surgeon to simulate the surgery, select or design an implant that more closely matches the contours of the patient's actual bone, and select or design surgical instruments and guide tools that are adapted specifically for repairing the bone of a particular patient. These tools are available intra-operatively to allow the surgeon to verify the diseased or damaged joint is cleared for implanting one or more prosthetic devices. This verification may result in a determination that an additional cut and/or excision is needed, such as cutting ligamentous attachments, scar tissue, tough capsule tissues, and other anatomical features for carrying out the surgical plan to completion. In addition, a surgeon may want to use these tools to view anatomical structure not visible at the surgical site. For example, the surgeon may want to obtain an intra-operative visualization of the medial view or posterior view of the surgical site while having direct visualization only in the anterior view.

In total ankle arthroplasty or total ankle replacement (TAR) surgery, the process may begin with a clinician making a series of incisions to expose a patient's ankle joint. The patient's distal tibial bone and proximal talus bone are exposed. The clinician performs various steps to align the tibia and talus, including, and not limited to, installing one or more guide pins using a physical guide to prepare the operating site for appropriate extraction of bone and aligned insertion of prosthetic components.

Prior to insertion of the prosthetic components, the area must be clear of bone, soft tissue, and other native anatomy to properly insert the prosthetic components. In performing the surgery, the patient's ankle joint is appropriately aligned to allow for effective extraction of one or more bone fragments and insertion of prosthetic components. During the surgery, to maintain appropriate alignment of the surgical site, there is limited mobility of the patient's ankle. As such, during the process of extracting the bone fragments, there are limits on the clinician's ability to visually confirm that all the intended bone fragments and native anatomy to be removed for insertion of the prosthetic components within the surgical site was effectively removed. Bone fragments or native anatomy remaining behind that should have been excised may lead to further complications in the surgical procedure, including adverse TAR surgery outcomes and future unforeseen outcomes for the patient's recovery. At the same time, extracting bone fragments and clearing debris from the surgical site is a delicate process, with the breaking of the lateral or medial malleoli of the distal tibia being an adverse outcome. If a clinician were to use too much force in removing bone fragments, the lateral and/or medial malleoli of the distal tibia may break. Alternatively, if a clinician were to gently attempt to shimmy the bone fragment out it may still impact the malleoli. Any sudden, unintended release of still-attached bone, soft tissue or native anatomy in the surgical site may result in sudden impact on the malleoli, for example.

To aid in proper and complete removal of bone fragments, soft tissue, debris and/or native anatomy expected to be removed from the surgical site without causing detrimental harm to the remaining patient anatomy, a clinician may, in accordance with one or more techniques of this disclosure, use an ultrasound device (e.g., an ultrasound probe) to scan portions of the surgical site not clearly visible due to the position of the patient's ankle. The ultrasound device may provide information regarding the sites of proper detachment of bone fragments, soft tissue, debris, and native anatomy and simultaneously provide information as to areas requiring additional clinician intervention to detach bone fragments, soft tissue, debris, and native anatomy still obstructing complete extraction to enable proper insertion of the prosthetic components at a later stage of the TAR surgery.

Thus, in accordance with one or more techniques of this disclosure, a processing system comprising one or more processors implemented in circuitry, may obtain ultrasound imaging data representing a non-viewable portion of a bone of a patient. The processing system may identify, based on the ultrasound imaging data, a soft tissue attachment point. The soft tissue attachment point may be a point on the non-viewable portion of the bone at which a soft tissue structure is attached to the bone. Furthermore, the processing system may cause an MR visualization device to display a virtual indicator superimposed on a viewable portion of the bone at a location corresponding to the soft tissue attachment point. Because the surgeon is able to view the virtual indicator, the surgeon may be able to locate and detach the corresponding soft tissue attachment point prior to attempting to extract the bone, thereby reducing the risk of harm to the patient while the surgeon is extracting the bone.

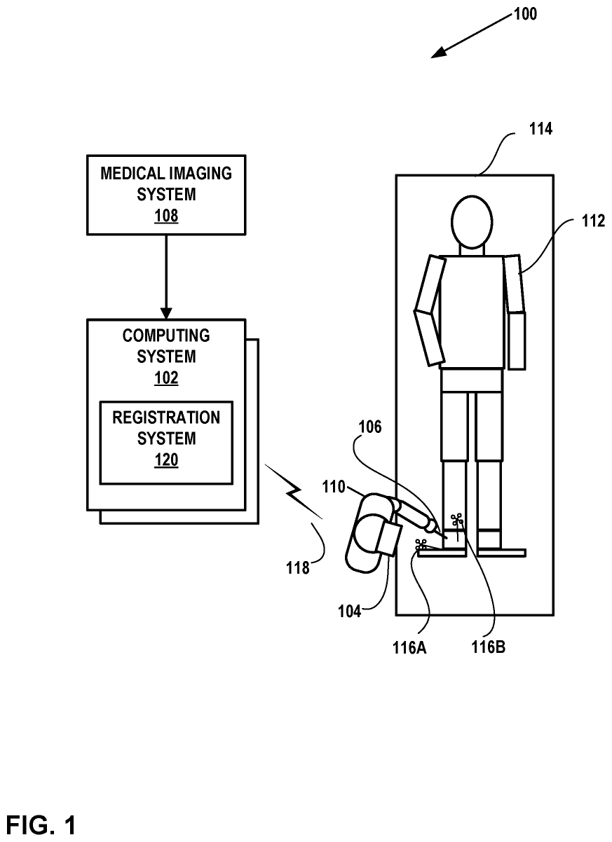

FIG. 1 is a conceptual diagram illustrating an example system 100 in which one or more techniques of this disclosure may be performed. In the example of FIG. 1, system 100 includes a computing system 102, a mixed realty (MR)-based visualization device 104, an ultrasound probe 106, and a medical imaging system 108. A clinician 110 is using ultrasound probe 106 to perform an examination on a patient 112 who is positioned on a table 114. Clinician 110 may be a surgeon, nurse, technician, medic, physician, or other type of medical professional or person. Clinician 110 and patient 112 do not form part of system 100. MR visualization device 104 may use markers 116A, 116B (collectively, “markers 116”) to determine a position of patient 112. Although the example of FIG. 1 shows clinician 110 performing the ultrasound examination on an ankle of patient 112, the techniques of this disclosure may be applicable with respect to other parts of the body of patient 112, such as a foot, shoulder, knee, hip, elbow, spine, wrist, hand, chest, and so on.

Normal ankle joint anatomy is formed by three bones joining together. The three bones include the tibia, the fibula, and the talus. The bony protrusions of the tibia and fibula over the ankle joint are the malleoli. The medial malleolus forms the base of the tibia at the inside of the ankle. The posterior malleolus also forms the base of the tibia at the back of the ankle. The lateral malleolus is the lower (i.e., distal) end of the fibula and felt on the outside of the ankle. The ankle joint allows up-and-down motion of the foot. The subtalar joint, located below the ankle joint, allows for side-to-side motion of the foot.

The total ankle arthroplasty or total ankle replacement (TAR) surgery is performed in sterile conditions in the operating room under general or spinal anesthesia. TAR surgery may begin with clinician 110 making a series of standard incisions to expose a patient's ankle joint. Clinician 110 may make a small cut at the anterior of the patient's ankle and expose the ankle joint. Clinician 110 may retract pertinent tissues and neurovascular structures from the site of the operation. The patient's distal tibial bone and proximal talus bone are exposed. Clinician 110 may remove periosteum and scar tissue from the distal tibia and the proximal talus in preparation of the surgical site for extraction of bone. Clinician 110 may use specialized surgical instruments to cut distal tibia and proximal talus for extraction of bone to accommodate a new prostheses mechanism, which may include the INFINITY TAR product available from Stryker Corp.

In general, during the surgical procedure, clinician 110 performs the ultrasound examination by positioning ultrasound probe 106 on the skin of patient 112. Ultrasound probe 106 generates ultrasonic waves and detects returning ultrasonic waves. The returning ultrasonic waves may include reflections of the ultrasonic waves generated by ultrasound probe 106. Ultrasound probe 106 may generate data based on the detected returning ultrasonic waves. The data generated by ultrasound probe 106 may be processed to generate ultrasound images, e.g., by ultrasound probe 106, computing system 102, or another device or system. In some examples, ultrasound probe 106 is a linear array ultrasound probe that detects returning ultrasound waves along a single plane oriented orthogonal to the direction of travel of the ultrasonic waves. A linear array ultrasound probe may generate 2D ultrasound images. In some examples, ultrasound probe 106 may be configured to perform 3D ultrasound, e.g., by rotating a linear array of ultrasound transducers.

MR visualization device 104 may use various visualization techniques to display MR visualizations to clinician 110. A MR visualization may comprise one or more virtual objects that are viewable by a user at the same time as real-world objects. Thus, what clinician 110 sees is a mixture of real and virtual objects.

MR visualization device 104 may comprise various types of devices for presenting MR visualizations. For instance, in some examples, MR visualization device 104 may be a Microsoft HOLOLENS™ headset, such as the HOLOLENS 2 headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses. In some examples, MR visualization device 104 may be a holographic projector, head-mounted smartphone, special-purpose MR visualization device, or another type of device for presenting MR visualizations. In some examples, MR visualization device 104 includes a head-mounted unit and a backpack unit that performs at least some of the processing functionality of MR visualization device 104. In other examples, all functionality of MR visualization device 104 is performed by hardware residing in a head-mounted unit. Discussion in this disclosure of actions performed by system 100 may be performed by computing system 102, MR visualization device 104, or a combination of the one or more computing devices and MR visualization device 104.

Augmented reality (AR) is similar to MR in the presentation of both real-world and virtual elements, but AR generally refers to presentations that are mostly real, with a few virtual additions to “augment” the real-world presentation. For purposes of this disclosure, MR is considered to include AR. For example, in AR, parts of the user's physical environment that are in shadow can be selectively brightened without brightening other areas of the user's physical environment. This example is also an instance of MR in that the selectively-brightened areas may be considered virtual objects superimposed on the parts of the user's physical environment that are in shadow.

Visualization tools are available that utilize patient image data to generate three-dimensional models of bone contours to facilitate preoperative planning for joint repairs and replacements. These tools allow surgeons to design and/or select surgical guides and prosthetic components that closely match the patient's anatomy. These tools can improve surgical outcomes by customizing a surgical plan for each patient. An example of such a visualization tool for shoulder joint repairs is the BLUEPRINT™ system available from Stryker Corporation. The BLUEPRINT™ system provides the surgeon with two-dimensional planar views of the bone repair region as well as a three-dimensional virtual model of the repair region. The surgeon can use the BLUEPRINT™ system to select, design or modify appropriate prosthetic components, determine how best to position and orient the prosthetic components and how to shape the surface of the bone to receive the prosthetic components, and design, select or modify surgical guide tool(s) or instruments to carry out the surgical plan. The information generated by the BLUEPRINT™ system may be compiled in a preoperative surgical plan for the patient that is stored in a database at an appropriate location (e.g., on a server in a wide area network, a local area network, or a global network) where it can be accessed by the surgeon or other care provider, including before and during the actual surgery.

Processing circuitry performing computing tasks of system 100 may be distributed among one or more computing devices of computing system 102, MR visualization device 104, ultrasound probe 106, and/or other computing devices. Furthermore, in some examples, system 100 may include multiple MR visualization devices. Computing devices of computing system 102 may include server computers, personal computers, smartphones, tablet computers, laptop computers, and other types of computing devices. Computing devices of computing system 102 may communicate with MR visualization device 104 via one or more wired or wireless communication links. In the example of FIG. 1, a lightning bolt 118 represents a wireless communication link between computing devices of computing system 102 and MR visualization device 104.

In accordance with one or more techniques of this disclosure, system 100 may obtain reference imaging data depicting one or more bones of patient 112. Medical imaging system 108 may generate the reference imaging data. Medical imaging system 108 may generate the reference imaging data prior to the ultrasound examination. In some examples, medical imaging system 108 generates computed tomography (CT) data. In other examples, medical imaging system 108 may generate magnetic resonance imaging (MRI) data or other types of medical images. Example types of reference imaging data may include one or more CT images, MRI images, nuclear magnetic resonance (NMR) images, and so on. In still other examples, medical imaging system 108 may generate the referencing imaging data at varying stages of the surgical procedure based on the preferences of clinician 110.

Additionally, system 100 may obtain ultrasound imaging data representing a non-viewable portion of a bone of patient 112. System 100 may generate the ultrasound imaging data based on data from ultrasound probe 106 or ultrasound probe 106 may generate the ultrasound imaging data. The ultrasound imaging data may include an ultrasound image. In some examples, system 100 may obtain an ultrasound image based on estimated distances to structures within patient 112. For instance, the ultrasound image may include pixels corresponding to distances from a transducer of ultrasound probe 106. In a typical ultrasound image, pixels corresponding to distances of structures that reflect ultrasonic waves are shown in white while other pixels remain dark. The structures represented in the ultrasound image may include soft tissue structures and bone.

MR visualization device 104 may perform a Simultaneous Localization and Mapping (SLAM) algorithm to determine a position of MR visualization device 104 and real-world objects in a coordinate system. This coordinate system may be referred to as a real-world coordinate system. Coordinates in the real-world coordinate system may be referred to as “real-world coordinates.” The real-world objects may include one or more bones of patient 112, registration markers 116, surgical tools, ultrasound probe 106, and other physical objects. Registration markers 116 may help MR visualization device 104 determine real-world coordinates of real-world objects within the real-world coordinate system. For example, registration markers 116 may aid in determining real-world coordinates for the position of patient 112 and the position of one or more bones at the surgical site.

In the example of FIG. 1, computing system 102 includes registration system 120. Registration system 120 may register a virtual coordinate system of the reference imaging data with the real-world coordinate system of MR visualization device 104. Additionally, registration system 120 may register a virtual coordinate system of the ultrasound-based imaging data with the real-world coordinate system. In some examples, registration system 120 may indirectly register the virtual coordinate system of the ultrasound-based imaging data with the real-world coordinate system by registering the virtual coordinate system of the ultrasound-based imaging data with the virtual coordinate system of the reference imaging data (which is registered with the real-world coordinate system). Indirectly registering the virtual coordinate system of the ultrasound-based imaging data with the real-world coordinate system by registering each with the virtual coordinate system of the reference imaging data may be advantageous because the ultrasound-based imaging data and imaging data generated MR visualization device 104 might not represent the same parts of the bones, which may make registration difficult, impossible, or imprecise. However, the reference imaging data may represent all relevant parts of the bones, thereby allowing for better registration of the virtual coordinate system of the ultrasound-based imaging data and real-world coordinate system with the virtual coordinate system of the reference imaging data. In this way, computing system 102 may be able to determine coordinates based on how positions in the ultrasound-based imaging data, the reference imaging data, and the real-world coordinates relate to one another.

In some examples, to register the virtual coordinate system of the ultrasound-based imaging system with the virtual coordinate system of the reference imaging data, registration system 120 may analyze the ultrasound-based imaging data to identify one or more structures represented in the ultrasound-based imaging data that have the same profiles as a bone represented in the reference imaging data.

Moreover, if registration system 120 finds the match, registration system 120 may determine real-world coordinates for the actual bone of patient 112. Registration system 120 may determine the real-world coordinates of the bone based on the distance of the bone from ultrasound probe 106 (as determined using the ultrasound image) and the real-world coordinates of ultrasound probe 106. Points on the bone as depicted in the reference data may be defined by a virtual coordinate system. Because system 100 is able to match a structure represented in the reference data with a structure represented in the ultrasound image, system 100 is therefore able to determine a relationship between the virtual coordinate system of the reference data and the real-world coordinate system. In other words, system 100 may generate registration data that registers the reference data with the real-world coordinate system.

Furthermore, in accordance with one or more techniques of this disclosure, system 100 may identify, based on the ultrasound imaging data, one or more soft tissue attachment points. In some examples, system 100 may determine locations in the virtual coordinate system of the ultrasound imaging data of the soft tissue attachment points. Each of the soft tissue attachment points is point on a non-viewable portion of the bone at which a soft tissue structure is attached to the bone. System 100 may cause MR visualization device 104 to display virtual indicators superimposed on a viewable portion of the bone at a location corresponding to the soft tissue attachment point. In other words, the virtual indicators may provide soft tissue location information to clinician 110 regarding areas of attachment remaining of the bone fragment of patient 112 to be resected. For example, the virtual indicators may provide clinician 110 the location of residual soft tissue attachments remaining after a clinician 110 has made the cut to the actual bone of patient 112. In some examples, the virtual indicators may provide clinician 110 with information of the location of attachments not viewable merely by visual inspection of the surgical site in the anterior view.

System 100 may use the registration data to determine the locations at which MR visualization device 104 is to display the virtual indicators. As noted above, registration system 120 may use reference imaging data to register the ultrasound-based imaging data with the real-world coordinate system. In general, the referencing imaging data provides a more complete and precise representation of bones and soft tissue anatomy than may be generated by ultrasound probe 106 and MR visualization device 104. In general, system 100 can predict the positions of various soft tissue structures based on the shapes and positions of the bones represented in the reference data. Thus, because both the reference imaging data and ultrasound-based imaging data are registered with the real-world coordinate system, system 100 may generate the virtual indicators representing locations of remaining soft tissue attachments as guidance or verification to clinician 110 regarding how the bone fragment to be resected remains attached at locations represented by the generated virtual indicators. For instance, the virtual indicators may inform clinician 110 to consider performing additional cuts to detach the soft tissue structure at the indicated location to detach the bone fragment.

System 100 may update the virtual indicators being displayed on the MR visualization device 104 after clinician 110 has attempted to detach one or more of the soft tissue attachment points. Thus, clinician 110 may obtain feedback on locations of remaining soft tissue attachment points to a bone fragment to be resected. In this way, clinician 110 may make an assessment and consider additional cuts/excisions to detach soft tissue structures from the bone fragment.

In some examples, system 100 may generate a virtual model of a soft tissue structure of patient 112 based on ultrasound imaging data regarding the soft tissue structure. System 100 may segment the ultrasound images to isolate parts of the ultrasound images that correspond to the soft tissue structure. In some examples, system 100 may use a machine learning (ML) model based on a computer vision technique (e.g., a convolutional neural network) to segment the ultrasound images to isolate the parts of the ultrasound images that correspond to the soft tissue structure. System 100 may then process the parts of the ultrasound images that correspond to the soft tissue structure to form the virtual model of the soft tissue structure. MR visualization device 104 may output the virtual model of the soft tissue structure so that the virtual model of the soft tissue structure appears to clinician 110 to be superimposed on patient 112 at an actual location of the soft tissue structure. MR visualization device 104 may also output virtual models of one or more bones of patient 112 so that the virtual bones of the bones appear to clinician 110 to be superimposed on patient 112 at actual locations of the bones of patient 112. In this way, clinician 110 may be able to easily comprehend the locations of hidden soft tissue structures and bones of patient 112. Being able to view virtual models of the soft tissue structure and bones on MR visualization device 104 may be especially valuable during a surgery.

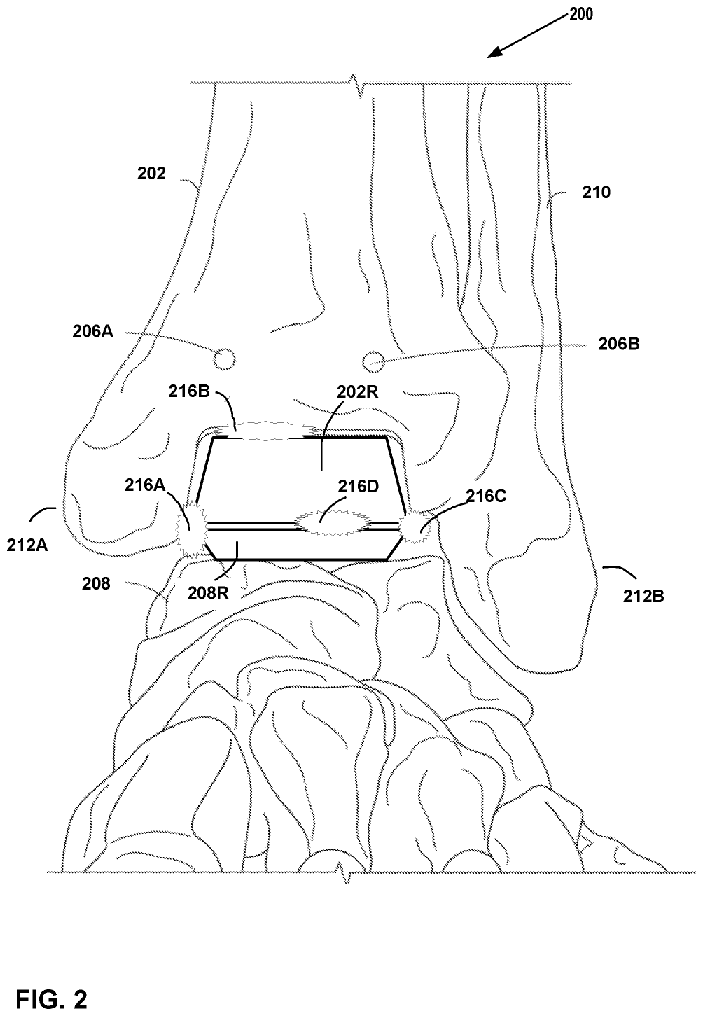

FIG. 2 is a conceptual diagram anterior view 200 of an example left ankle ready for extracting a tibial bone fragment 202R and a talar bone fragment 208R during a TAR surgery, in accordance with one or more techniques of this disclosure. During the course of the TAR surgery, clinician 110 makes incisions and/or cuts to prepare a tibia 202 of patient 112, followed by extracting tibial bone fragment 202R. Additionally, clinician 110 may make incisions and/or cuts to prepare talus 208, followed by extracting talar bone fragment 208R. Clinician 110 may then place tibial and/or talus prosthetic components, confirm fit of the tibial and/or talus prosthetic components, and confirm appropriate movement of the ankle having the prosthetic components. Clinician 110 may perform several work steps on one or both of tibia 202 and/or talus 208 to prepare tibia 202 and/or talus 208 to receive the prosthetic components. The work steps may include installing guide pins at locations 206A and 206B into tibia 202. Another additional work step may include confirming the integrity of the posterior malleolus of the tibia (not shown), a lateral malleolus 212B of fibula 210, and a medial malleolus 212A of tibia 202.

Furthermore, as part of preparing tibia 202, fibula 210, and talus 208 to receive the prosthetic components, clinician 110 steps may visually inspect the surgical site to confirm that tibial bone fragment 202R and talar bone fragment 208R are fully detached from tibia 202, talus 208, and any soft tissue structures. In accordance with one or more techniques of this disclosure, clinician 110 may use MR visualization device 104 in conjunction with ultrasound probe 106 to visually inspect of the sides and back of the surgical site to confirm appropriate detachment of bone and soft tissue structures.

System 100 (FIG. 1) may obtain ultrasound imaging data representing non-viewable areas of a bone (e.g., tibia 202, talus 208, fibula 210, etc.) at the surgical site. The ultrasound imaging data may include data generated by ultrasound probe 106 (FIG. 1) or data based on data generated by ultrasound probe 106. System 100 may identify, based on the ultrasound imaging data, soft tissue attachment points. The soft tissue attachment points may be points or areas where soft tissue structures, such as tendons, ligaments, cartilage, scar tissue, tough capsule tissue, or blood vessels, for example, are attached to bones. The soft tissue attachment points may be at locations in non-viewable medial and/or posterior side of the surgical site when the surgical site is viewed from the anterior side of the surgical site. In some examples, system 100 may identify types of soft tissue based on the ultrasound imaging data.

Registration system 120 may directly or indirectly register a real-world bone (e.g., tibia 202, talus 208, fibula 210) of patient 112 with an ultrasound-based 3D model. Registration system 120 may generate the ultrasound-based 3D model based on data received from ultrasound probe 106. The ultrasound-based 3D model may be a mesh, point cloud, voxel-based 3D image, or other 3D representation of one or more bones of patient 112. In some examples, the ultrasound-based 3D model may also represent soft tissue structures. Registration system 120 may register a virtual coordinate system of the ultrasound-based 3D model with the real-world coordinate system. The virtual coordinate system of the ultrasound-based 3D model is a coordinate system used to express positions within the ultrasound-based 3D model. Additionally, registration system 120 may register the real-world bone with a 3D reference bone model (e.g., reference imaging data, such as a CT-based bone model). In this way, computing system 102 is able to determine corresponding locations in each of the ultrasound-based 3D model, the 3D reference bone model, and the real-world bone of patient 112.

System 100 may identify soft tissue attachment points in the ultrasound-based 3D model. For instance, system 100 may apply an ML model that segments the ultrasound-based 3D model. In other words, the ML model may determine parts of the ultrasound-based 3D model that represent bone and parts of the ultrasound-based 3D model that represent various soft tissue structures. System 100 may identify locations where soft tissue structures connect with bone as soft tissue attachment points.

After completing registration and identifying the soft tissue attachment points, computing system 102 may cause MR visualization device 104 to display virtual indicators superimposed on viewable portions of the bones (e.g., tibia 202, talus 208, fibula 210) of patient 112 at locations corresponding to the soft tissue attachment points. In other words, MR guidance indicating soft tissue attachment locations may be generated. In the example of FIG. 2, MR visualization device 104 displays virtual indicators 216A, 216B, 216C, 216D (collectively, “virtual indicators 216”) at locations corresponding to soft tissue attachment points. MR visualization device 104 may display virtual indicators 216 overlaid (e.g., superimposed) on and registered to the real anatomy of patient 112. In such examples, only some of the bones shown in FIG. 2 are actually visible to clinician 110. In some examples, MR visualization device 104 may display virtual indicators 216 and models of bones in a virtual display panel that is not overlaid on the real anatomy of patient 112. For instance, in such examples, the virtual display panel may appear at a corner or side of the field of vision of clinician 110. The virtual display panel may be a 2D panel analogous to a television screen or computer monitor.

Soft tissue attachment points may limit the ability of clinician 110 to extract tibial bone fragment 202R and/or talar bone fragment 208R. Visual inspection of anterior view 200 of the exposed ankle at the surgical site by clinician 110 without the aid of MR visualization device 104 may be sufficient to confirm clearance of the tibial bone fragment 202R and/or talar bone fragment 208R. However, visual inspection in anterior view 200 of the exposed ankle at the surgical site may not provide complete posterior, lateral, and medial views with depth information to verify the locations of the soft tissue attachment points. By displaying virtual indicators 216 indicating locations of the soft tissue attachment points, clinician 110 may have a better understanding of where soft tissue structures may still be attached to non-viewable posterior, lateral, and medial surfaces of tibial bone fragment 202R and talar bone fragment 208R.

After clinician 110 has made incisions and/or cuts to prepare tibia 202 and before extracting tibial bone fragment 202R, clinician 110 may use virtual indicators 216 to determine the locations of the soft tissue attachment points. Clinician 110 may then attempt to detach the soft tissue attachment points prior to extraction of tibial bone fragment 202R. For instance, clinician 110 may perform additional incisions and/or cuts to detach a tendon, a ligament, a muscle, cartilage, scar tissue, tough capsule tissue, or a blood vessel, or other soft tissue structure. Clinician 110 may use a hooked tool, e.g., a posterior capsule release tool, to scrape away soft tissue attachment points from the posterior, lateral, or medial locations of the bone fragments.

After attempting to detach one or more soft tissue attachment points on tibial bone fragment 202R, clinician 110 may use ultrasound probe 106 again to scan the posterior, lateral, and/or medial portions of the ankle of patient 112. Computing system 102 may analyze the data generated by ultrasound probe 106 to identify updated locations of remaining soft tissue attachment points. MR visualization device 104 may then present an updated MR guidance, (e.g., virtual indicators), that MR visualization device 104 superimposes on the surgical site. The updated MR guidance indicates the locations of the remaining soft tissue attachment points. The use of MR visualization device 104 and ultrasound probe 106 may be repeated until clinician 110 is satisfied with the detachment of soft tissue and clearance of tibial bone fragment 202R for extraction of resected bone. Afterward, clinician 110 may extract tibial bone fragment 202R. During the extraction of tibial bone fragment 202R, clinician 110 may or may not use the aid of MR visualization device 104. Clinician 110 and system 100 may do the same for talar bone fragment 208R.

In some examples, features of virtual indicators 216 may indicate depths of soft tissue attachment points relative to the anterior view 200 of the surgical site. For instance, in the example of FIG. 2, virtual indicator 216A is smaller in size in comparison to virtual indicator 216D. The size of a virtual indicator may correspond to the distance from MR visualization device 104 to the soft tissue attachment point corresponding to the virtual indicator. The soft tissue attachment corresponding to virtual indicator 216D may be closer to MR visualization device 104. In contrast, virtual indicator 216A may correspond to another soft tissue attachment point may be further away from the viewable anterior view 200 of the surgical site. In some examples, the sizes of a virtual indicators 216 may correspond to the sizes of the corresponding soft tissue attachment points. For example, virtual indicator 216A has a shorter radius than virtual indicator 216B. This variability may correspond to the length of the soft tissue attachment points corresponding to virtual indicators 216A, 216B. In some examples, virtual indicators 126 may be color and/or texture coded to indicate cross-sectional thicknesses or depths (e.g., distance an anterior-posterior direction), tissue types, or other aspects of soft tissue attachment points.

In some examples, MR visualization device 104 may present information in addition to virtual indicators 216. For instance, MR visualization device 104 may also display information that indicates the types of soft tissue structures attached to the bones at the soft tissue attachment points corresponding to virtual indicators 216. In some examples, MR visualization device 104 may also display images, features, information, or data in combination with real-world information. In some examples, MR visualization device 104 may display images from data collected by ultrasound probe 106. For instance, MR visualization device 104 may display an ultrasound image that shows locations of soft tissue attachment points.

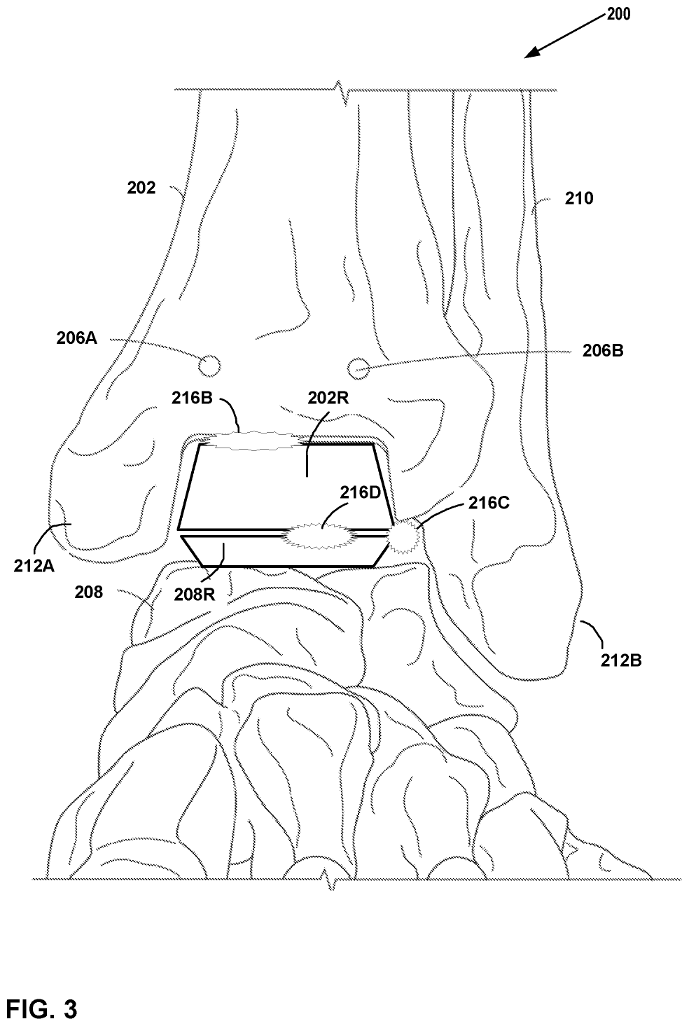

FIG. 3 is a conceptual diagram illustrating an MR visualization of virtual indicators in the surgical site, in accordance with one or more techniques of this disclosure. After viewing virtual indicators 216 shown in FIG. 2, clinician 110 may detach one or more of the soft tissue attachment points corresponding to virtual indicators 216. For instance, clinician 110 may use a hooked tool to detach soft tissue attachment points on the posterior side of tibia 202, fibula 210 or talus 208. After detaching one or more soft tissue attachment points, clinician 110 may use ultrasound probe 106 again to obtain updated ultrasound imaging data. Computing system 102 may perform the same process as described elsewhere in this disclosure (e.g., with respect to FIG. 2) to identify, based on the updated ultrasound imaging data, remaining soft tissue attachment points. Computing system 102 may cause MR visualization device 104 to display an updated set of virtual indicators superimposed on a viewable portion of the bones of patient 112 at locations corresponding to the updated set of soft tissue attachment points. As shown in the example of FIG. 3, virtual indicator 216A is no longer shown because clinician 110 has successfully detached the soft tissue attachment point corresponding to virtual indicator 216A.

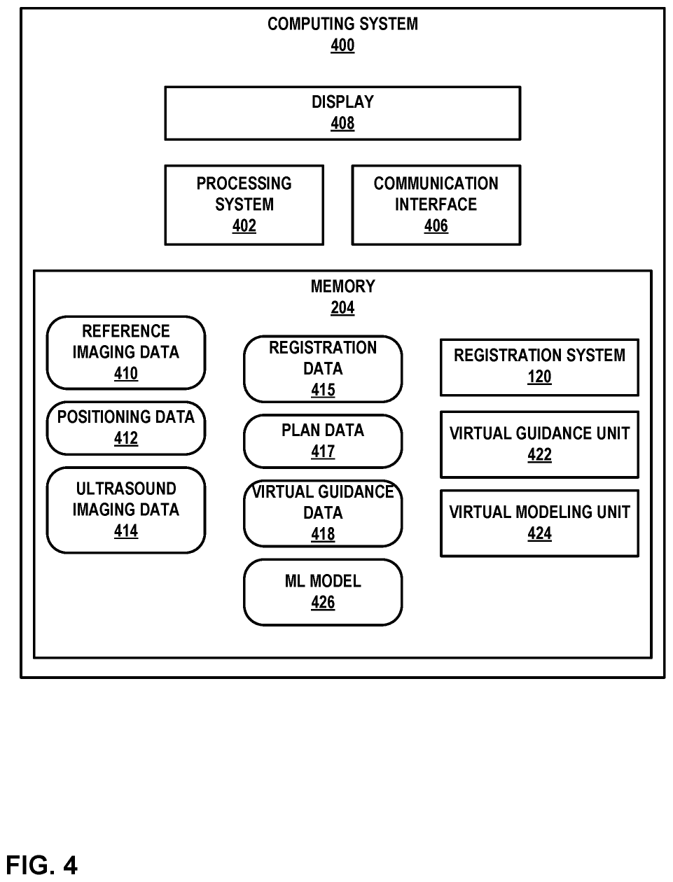

FIG. 4 is a conceptual diagram illustrating an example computing system 400 in accordance with one or more techniques of this disclosure. Components of computing system 400 of FIG. 4 may be included in computing system 102 (FIG. 1), MR visualization device 104, or ultrasound probe 106. In the example of FIG. 4, computing system 400 includes processing system 402, memory 404, a communication interface 406, and a display 408.

Examples of processing system 402 include one or more microprocessors, digital signal processors (DSPs), application specific integrated circuits (ASICs), field programmable gate arrays (FPGAs), hardware, or any combinations thereof. In general, processing system 402 may be implemented as fixed-function circuits, programmable circuits, or a combination thereof. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, one or more of the units may be distinct circuit blocks (fixed-function or programmable), and in some examples, the one or more units may be integrated circuits.

Processing system 402 may include arithmetic logic units (ALUs), elementary function units (EFUs), digital circuits, analog circuits, and/or programmable cores, formed from programmable circuits. In examples where the operations of processing system 402 are performed using software executed by the programmable circuits, memory 404 may store the object code of the software that processing system 402 receives and executes, or another memory within processing system 402 (not shown) may store such instructions. Examples of the software include software designed for surgical planning. Processing system 402 may perform the actions ascribed in this disclosure to computing system 400.

Memory 404 may store various types of data used by processing system 402, Memory 404 may include any of a variety of memory devices, such as dynamic random-access memory (DRAM), including synchronous DRAM (SDRAM), magnetoresistive RAM (MRAM), resistive RAM (RRAM), or other types of memory devices. Examples of display 408 include a liquid crystal display (LCD), a plasma display, an organic light emitting diode (OLED) display, or another type of display device.

Communication interface 406 that allows computing system 400 to output data and instructions to and receive data and instructions from MR visualization device 104. medical imaging system 108, or other device via one or more communication links or networks. Communication interface 406 may include hardware circuitry that enables computing system 400 to communicate (e.g., wirelessly or using wires) to other computing systems and devices, such as MR visualization device 104. Example networks may include various types of communication networks including one or more wide-area networks, such as the Internet, local area networks, and so on. In some examples, the network may include wired and/or wireless communication links.

In the example of FIG. 4, memory 404 stores reference imaging data 410, positioning data 412, ultrasound imaging data 414, registration data 415, plan data 417, virtual guidance data 418, and a ML model 426. Additionally, memory 404 stores registration system 120, a virtual guidance unit 422, and a virtual modeling unit 424. In other examples, memory 404 may store more, fewer, or different types of data or units. Moreover, the data and units illustrated in the example of FIG. 4 are provided for purposes of explanation and may not represent how data is actually stored or how software is actually implemented. Registration system 120, virtual guidance unit 422, and virtual modeling unit 424 may comprise instructions that are executable by processing system 402. For ease of explanation, this disclosure may describe registration system 120, virtual guidance unit 422, virtual modeling unit 424 as performing various actions when processing system 402 executes instructions of registration system 120, virtual guidance unit 422, virtual modeling unit 424.

In general, reference imaging data 410 includes previously obtained data depicting one or more bones of patient 112. For example, reference imaging data 410 may include one or more CT images of a bone. In some examples, reference imaging data 410 may include a 3-dimensional (3D) model of a bone. In other words, reference imaging data 410 may include a 3D reference bone model. The 3D reference bone model may be generated based on a plurality of CT images. Reference imaging data 410 may be generated by medical imaging system 108 or based on data from medical imaging system 108. In some examples, computing system 400 may receive reference imaging data 410 from another source.

Positioning data 412 may include data indicating locations of ultrasound probe 106, patient 112, and/or other real-world objects. Computing system 400 may obtain positioning data 412 based on one or more sensors, such as depth sensors or cameras, located on MR visualization device 104 and/or other devices. Ultrasound imaging data 414 may include ultrasound images or data generated by ultrasound probe 106. In some examples, computing system 400 may use the data generated by ultrasound probe 106 to generate an ultrasound-based 3D model. Plan data 417 may include data related to a plan for a medical task. For instance, plan data 417 may indicate which soft tissue structures are relevant for the medical task.

Registration system 120 may generate first registration data that registers a virtual coordinate system of the ultrasound-based 3D model (ultrasound imaging data 414) with a virtual coordinate system of the 3D reference bone model (reference imaging data 410). Additionally, registration system 120 may generate second registration data that registers a coordinate system of MR visualization device 104 (e.g., a “real-world coordinate system”) with the virtual coordinate system of the 3D reference bone model. Registration data 415 may include the first registration data and the second registration data.

Example methods of generating registration data may include the Iterative Closest Point (ICP) algorithm and the Nelder-Mead algorithm. As part of performing a registration process, registration system 120 may generate a first point cloud and a second point cloud. The first point cloud and the second point cloud may correspond to different ones of the ultrasound-based 3D model, the 3D reference bone model, and model of real-world objects. MR visualization device 104 may generate the model of real-world objects, e.g., based on data from depth sensors. The ICP algorithm may determine registration data (e.g., a combination of translational and rotational parameters) that minimize the sum of distances between corresponding points in the first-and second-point clouds. For example, consider an example where landmarks corresponding to points in the first point cloud are at coordinates A, B, and C and the same landmarks corresponding to points in the second point cloud are at coordinates A′, B′, and C′. In this example, the ICP algorithm determines a combination of translational and rotational parameters that minimizes ΔA+ΔB+ΔC, where ΔA is the distance between A and A′, ΔB is the distance between B and B′, and ΔC is the distance between C and C′. To minimize the sum of distances between corresponding landmarks in the first point cloud and the second point cloud, registration system 120 may perform following steps:
1. For each point of the first point cloud, determine a corresponding point in the second point cloud. The corresponding point may be the closest point in the second point cloud.
2. Estimate a combination of rotation and translation parameters using a root mean square point-to-point distance metric minimization technique that best aligns each point of the first point cloud to its corresponding point in the second point cloud.
3. Transform the points of the first point cloud using the estimated combination of rotation and translation parameters.
4. Iterate steps 1-3 using the transformed points of the first point cloud. The iteration may continue for a specific number of rounds, until changes between iterations decrease to a specific threshold, or another stopping criteria is reached.

In this example, after performing an appropriate number of iterations, registration system 120 may determine rotation and translation parameters that describe a spatial relationship between the original positions of the points in the first point cloud and the final positions of the points in the first point cloud. The determined rotation and translation parameters can therefore express a mapping between the first point cloud and the second point cloud.

In some examples, registration system 120 may register the first and second point clouds using a PointNetLK algorithm, e.g., as described in Aoki et al., “PointNetLK: Robust & Efficient Point Cloud Registration using PointNet,” 2019 IEEE/CVF Conference on Computer Vision and Pattern Recognition (CVPR), 2019, pp. 7156-7165, doi: 10.1109/CVPR.2019.00733.

In some examples, registration system 120 may register the first point cloud and the second point clouds using a process, such as a Nelder-Mead process, that optimizes a Dice Similarity Coefficient (DSC) metric. In an example that uses a Nelder-Mead process, registration system 120 may calculate a DSC that indicates a similarity between a hull defined by a first point cloud (or a 3D image, such as an ultrasound-based 3D bone image, a 3D reference bone image, 3D image generated by sensors of MR visualization device 104) and a hull defined by a second point cloud (or a 3D image, such as an ultrasound-based 3D bone image, the 3D reference bone image, 3D image generated by sensors of MR visualization device 104). Additionally, registration system 120 may apply transformations to the first point cloud to generate a set of 6 additional point clouds. The transformations may rotate and/or translate the first point cloud. Thus, the transformations may change the position of the first point cloud any of 6 degrees of freedom. Hence, a transformation applied to the first point cloud may be expressed as a 6-dimensional vector. The 6-dimensional vector may be considered “registration data.” In this way, each additional point cloud is associated with a 6-dimensional vector. Registration system 120 may calculate DSC metrics of the first point cloud and the additional point clouds. The DSC metric of a point cloud indicates a similarity between a hull defined by the first point cloud and a hull defined by the second point cloud. Thus, the DSC metric of a point cloud is associated with a location in a 6-dimensional vector space.

Furthermore, registration system 120 may determine an order of seven (7) locations in the 6-dimensional vector space (recall each location in the 6-dimensional vector space is associated with the DSC metric of the first point cloud or a transformed version of the first point cloud). The order of the 7 locations is based on the DSC metrics associated with the 7 locations. The 7 locations form a simplex in the 6-dimensional vector space. Registration system 120 may then determine whether a termination condition is reached. Registration system 120 may determine that the termination condition is reached if a standard deviation of the DSC metrics associated with the 7 locations of the simplex is less than a tolerance threshold. If the optimization process is not to terminate, registration system 120 may calculate a centroid location of all locations of the simplex except for a lowest-ordered location of the simplex.

As mentioned above, registration system 120 may generate first registration data that registers the virtual coordinate system of the ultrasound-based 3D model with a virtual coordinate system of the 3D reference bone model and may generate second registration data that registers a coordinate system of MR visualization device 104 (e.g., a “real-world coordinate system”) with the virtual coordinate system of the 3D reference bone model. Virtual guidance unit 422 may determine, based on the first registration data and the second registration data, a location in the coordinate system of MR visualization device 104 of the location corresponding to the soft tissue attachment point. For example, computing system 102 may determine, based on the first registration data, that a soft tissue attachment point at coordinates (x₁, y₁, z₁) in the virtual coordinate system of the ultrasound-based 3D model corresponds to a position at coordinates (x₂, y₂, z₂) in the virtual coordinate system of the 3D reference bone model. In this example, computing system 102 may further determine that the position at coordinates (x₂, y₂, z₂) in the virtual coordinate system of the 3D reference bone model correspond to coordinates (x₃, y₃, z₃) in the “real-world coordinate system (i.e., the coordinate system of MR visualization device 104). Thus, when MR visualization device 104 is pointed toward the location corresponding to the soft tissue attachment point (e.g., a location at coordinates (x₃, y₃, z₃)), MR visualization device 104 may display a virtual indicator superimposed on the viewable portion of the bone at the location corresponding to the soft tissue attachment point.

Virtual guidance unit 422 may cause MR visualization device 104 to generate the virtual guidance to clinician 110. For example, virtual guidance unit 422 may apply ML model 426 to identify soft tissue attachment points based on the ultrasound imaging data 414. Virtual guidance unit 422 may cause a MR visualization device 104 to display a virtual indicator superimposed on a viewable portion of the bone at a location corresponding to a soft tissue attachment point. Virtual modeling unit 424 may generate virtual models and, in some examples, may cause MR visualization device 104 to output the virtual models.

In some examples, registration system 120 may determine a physical location of ultrasound probe 106. Additionally, registration system 120 may generate, based on first ultrasound data generated by ultrasound probe 106, registration data that registers virtual locations on the bone of patient 112 as depicted in reference imaging data 410 with corresponding physical locations on the bone of patient 112. Virtual guidance unit 422 may generate virtual guidance data 418 based on reference imaging data 410, registration data 415, and the physical location of ultrasound probe 106 (e.g., positioning data 412). Virtual guidance data 418 may provide guidance to clinician 110 regarding how ultrasound probe 106 is positioned relative to a target position at which ultrasound probe 106 is able to generate ultrasound data that provides information regarding a soft tissue structure of the patient. For example, virtual guidance data 418 may instruct clinician 110 how to move ultrasound probe 106 so that ultrasound probe 106 is at a target position to generate ultrasound data that provides information regarding a soft tissue structure of patient 112.

Virtual guidance unit 422 may obtain an ultrasound image based on estimated distances to structures within patient 112. For instance, the ultrasound image may include pixels corresponding to distances from a transducer of ultrasound probe 106. In a typical ultrasound image, pixels corresponding to distances of structures that reflect ultrasonic waves are shown in white while other pixels remain dark.

In an example where ultrasound probe 106 is a linear array ultrasound probe, ultrasound probe 106 includes an array of transducers arranged in a single line along the detection plane of ultrasound probe 106. The transducers may be arranged in a fan shape. Thus, an ultrasound image generated by a linear array ultrasound probe may represent structures within a fan-shaped slice through patient 112 aligned with the detection plane. In some examples, a 3D ultrasound image of a cone-shaped section of patient 112 may be generated by rotating the linear array of transducers of ultrasound probe 106.

The structures represented in the ultrasound image may include soft tissue structures, for example, ligamentous attachments, scar tissue, tough capsule tissues, and other anatomical features, in addition to the bone. Virtual guidance unit 422 may analyze the ultrasound image to identify a structure represented in the ultrasound image that has the same profile as a structure represented in the reference data. For instance, virtual guidance unit 422 may analyze the ultrasound image to identify a curve of a structure represented in the ultrasound image. Virtual guidance unit 422 may then attempt to match that curve to a curve of a structure represented in the reference data. If virtual guidance unit 422 finds a match, the structure represented in the ultrasound image is likely to be the structure represented in the reference data.

Moreover, if virtual guidance unit 422 finds the match, virtual guidance unit 422 may determine real-world coordinates for the structure. Virtual guidance unit 422 may determine the real-world coordinates of the bone based on the distance of the bone from ultrasound probe 106 (as determined using the ultrasound image) and the real-world coordinates of ultrasound probe 106. Points on the bone as depicted in the reference data may be defined by a virtual coordinate system. Because virtual guidance unit 422 is able to match a curve of the bone represented in the reference data with a curve of the bone represented in the ultrasound image, virtual guidance unit 422 is therefore able to determine a relationship between the virtual coordinate system of the reference data and the real-world coordinate system. In other words, virtual guidance unit 422 may generate registration data that registers the reference data with the real-world coordinate system.

Furthermore, virtual guidance unit 422 may determine a spatial relationship between ultrasound probe 106 and the bone in addition to soft tissue structures, for example, ligamentous attachments, scar tissue, tough capsule tissues, and other anatomical features. In other words, virtual guidance unit 422 may determine where ultrasound probe 106 is in relation to soft tissue structures and the actual bone of patient 112.

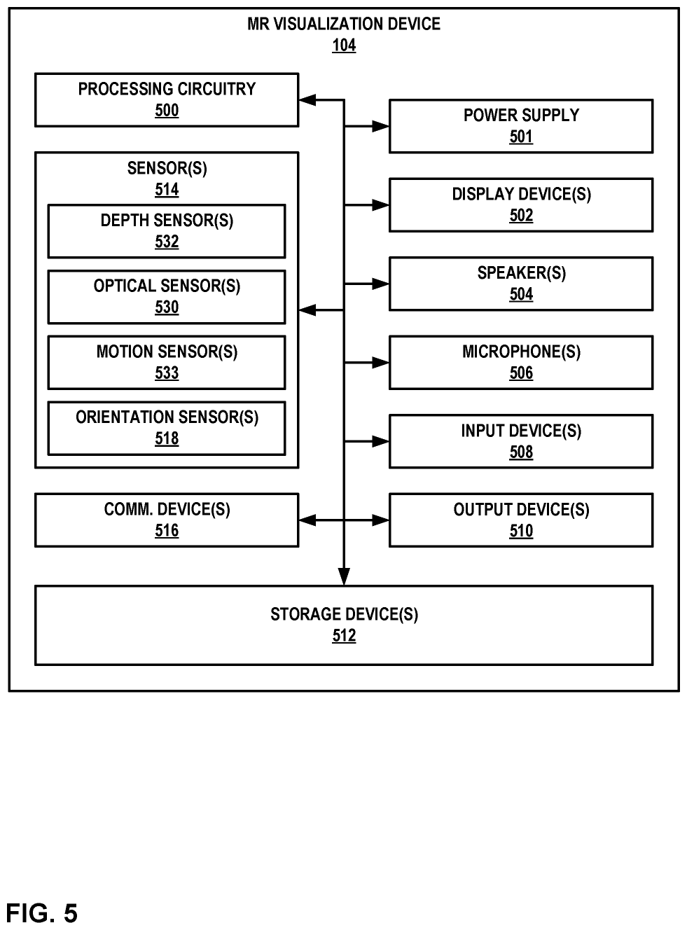

FIG. 5 is a block diagram illustrating example components of MR visualization device 104 for use in an MR system. In the example of FIG. 5, MR visualization device 104 includes processing circuitry 500, a power supply 501, one or more display devices 502, one or more speakers 504, one or more microphones 506, one or more input devices 508, one or more output devices 510, one or more storage devices 512, one or more sensors 514, and one or more communication devices 516. Power supply 501 may supply electrical energy to processing circuitry 500, display device(s) 502, speaker(s) 504, microphone(s) 506, input device(s) 508, output device(s) 510, storage device(s) 502, sensor(s) 514, communication device(s) 516, and/or other components of MR visualization device 104. Processing circuitry 500 may be implemented in accordance with any of the examples provided elsewhere in this disclosure with respect to processing system 402 (FIG. 4).

In the example of FIG. 5, sensor(s) 514 may include depth sensor(s) 532, optical sensor(s) 530, motion sensor(s) 533, and orientation sensor(s) 518. Optical sensor(s) 530 may include cameras, such as Red-Green-Blue (RGB) video cameras, infrared cameras, or other types of sensors that form images from light. In other examples, sensor(s) 514 may include more, fewer, or different sensors.

Display device(s) 502 may display imagery, such as MR visualizations, to a user, such as clinician 110. In some examples, display device(s) 502 may include a screen. For example, as discussed, display device(s) 502 may include see-through holographic lenses, in combination with projectors, that permit a user to see real-world objects, in a real-world environment, through the lenses, and also see virtual 3D holographic imagery projected into the lenses and onto the user's retinas, e.g., by a holographic projection system. In this example, virtual 3D holographic objects may appear to be placed within the real-world environment. In some examples, display device(s) 502 include one or more display screens, such as LCD display screens, OLED display screens, and so on. The user interface may present virtual images of details of the virtual surgical plan for a particular patient.

In some examples, a user may interact with and control MR visualization device 104 in a variety of ways. For example, microphone(s) 506, and associated speech recognition processing circuitry or software, may recognize voice commands spoken by the user and, in response, perform any of a variety of operations, such as selection, activation, or deactivation of various functions associated with surgical planning, intra-operative guidance, or the like. As another example, one or more cameras or other optical sensors 530 of sensor(s) 514 may detect and interpret gestures to perform operations as described above. As a further example, sensor(s) 514 may sense gaze direction and perform various operations as described elsewhere in this disclosure. In some examples, input device(s) 508 may receive manual input from a user, e.g., via a handheld controller including one or more buttons, a keypad, a touchscreen, joystick, trackball, and/or other manual input media, and perform, in response to the manual user input, various operations as described above.

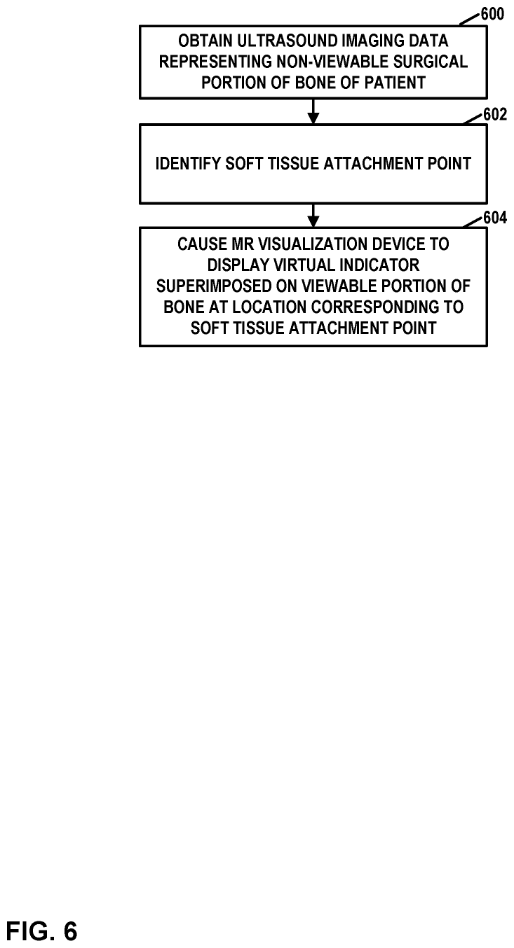

FIG. 6 is a flowchart illustrating an example operation of processing system 402, in accordance with one or more techniques of this disclosure. Briefly, as shown in the example of FIG. 6, computing system 102 obtains intraoperative ultrasound imaging data of non-viewable surgical sites of a patient, identifies still remaining soft tissue attachment points to bone fragments to be resected, registering location for the soft tissue attachment points to the bone fragments at the non-viewable surgical site, generating virtual indicators corresponding to the registered locations of the soft tissue attachments, and displaying via MR visualization device 104 the virtual indicators of the soft tissue attachment corresponding to a viewable surgical portion of the site.

Processing system 402 may obtain ultrasound imaging data representing a non-viewable portion of a bone of a patient (600). For example, in TAR surgery, a non-viewable portion may include the posterior, lateral, and/or medial portions of the ankle. As discussed above, in preparation of TAR surgery, the viewable portion of the bone of the patient is the anterior portion of the ankle anatomy including the tibia and talus. In this example, the bone may be tibial bone fragment 202R or talar bone fragment 208R. In other examples, the operation of FIG. 6 may be performed with respect to other anatomic areas of patient 112, such as the knee, elbow, shoulder, hip, spine, and so on, Thus, the bone may be one of: a femur, fibula, talus, vertebra, ilium, scapula, humerus, radius, ulna, or other bone of patient 112. The ultrasound imaging data may be obtained before or during the surgery. In some examples, obtaining the ultrasound imaging data comprises generating an ultrasound-based 3D model based on data received from ultrasound probe 106.

Processing system 402 may identify, based on the ultrasound imaging data, soft tissue attachment points (602). The soft tissue attachment points may include points on the non-viewable portion of the bone at which soft tissue structures are attached to bone. The soft tissue attachment points identified includes a tendon, a ligament, a muscle, cartilage, scar tissue, tough capsule tissue, or a blood vessel, for example. In some examples, processing system 402 applies ML model 426 to identifies the soft tissue attachment points based on the ultrasound imaging data.

In some examples, the ultrasound imaging data that contain striations and reflectivity (of the sound waves used by the ultrasound machine, which are represented on readouts by a shade of grey between black and white, proportional to the reflectivity) of various biological tissues in ultrasound. For example, boundaries between bone and ligaments are visible based upon how the sound waves reflect back to ultrasound probe 106. Attachment points are where the two materials meet and integrate,

Processing system 402 may identify a partial soft tissue attachment point based on ML model 426 by identifying areas of the ultrasound imaging data that correspond to boundaries and any deviation from expected “intact” and “detached” states. For example, ML model 426 may be implemented as a neural network having a convolutional neural network (CNN) branch and a reconstruction branch. The CNN branch includes a series of one or more convolutional layers (e.g., 3 convolutional layers) that reduce 3-dimensional ultrasound imaging data to a feature vector. The reconstruction branch includes a series of de-convolutional layers and pooling layers that transform the feature vector into a 3-dimensional output matrix having cells corresponding to locations in the 3-dimensional ultrasound imaging data. Data in the output matrix may contain classification data for the corresponding locations. For instance, the classification data in a cell of the output matrix may classify the corresponding location in the ultrasound imaging data as being or not being part of a soft tissue attachment point. In some examples, the classification data may indicate a type of the tissue at the corresponding location (e.g., cartilage, tendon, bone, etc.). In this example, processing system 402 may analyze the output matrix to identify groups of cells having data indicating that the corresponding locations are soft tissue attachment points. In this way, processing system 402 may identify the locations of the soft tissue attachment points in a 3-dimensional space.

ML model 426 may be trained (e.g., by computing system 104 or another computing system) based on training datasets derived from ultrasound imaging data of intact, detached, and partially detached structures of the anatomy of interest, which have been manually labeled. Procedure-specific training datasets may be generated using cadavers and collecting a new dataset iteratively throughout specific steps of the process (e.g., before, after cuts but before detachment step, 10% through detachment, 20% through detachment, etc., until complete).

Processing system 402 may register the soft tissue attachment points to a coordinate system, such as a virtual coordinate system of a bone reference model. In some examples, processing system 402 may generate first registration data that registers the virtual coordinate system of the ultrasound-based 3D model with a virtual coordinate system of the 3D reference bone model. Processing system 402 may generate second registration data that registers a coordinate system of MR visualization device 104 with the virtual coordinate system of the 3D reference bone model. Processing system 402 determines a location in the coordinate system of MR visualization device 104 (e.g., a “real-world coordinate system”) of the location corresponding to the soft tissue attachment point, based on the first and second registration data.

Processing system 402 may cause MR visualization device 104 to display a virtual indicator (e.g., one of virtual indicators 216) superimposed on a viewable portion of the bone at a location corresponding to the soft tissue attachment point (604). The non-viewable portion of the bone and the viewable portion of the bone may be on opposite sides of the bone. The non-viewable portion of the bone may be an area on a first side of the bone, the viewable portion of the bone may be an area on a second side of the bone opposite the first side of the bone at which an incision to access the bone has been made, For example, in a TAR surgery, the surgical site of the ankle exposes the anterior view of the tibia and talus as the viewable surgical portion. The non-viewable surgical portion is the posterior, medial, and/or lateral views of the tibia and talus. Thus, in this example, the bone may be a tibia, the non-viewable portion of the bone is an area on a posterior side of the tibia, the viewable portion of the bone is an area on an anterior side of the tibia at which an incision to access the tibia has been made.

In some examples where a surgical procedure involves removing portions of a bone, such as osteophytes, protrusions, etc., processing system 402 may cause MR visualization device 104 to display one or more virtual indicators superimposed on the viewable portion of the bone at a location corresponding to target bone tissue on the non-viewable portion of the bone or another bone of the patient. These virtual indicators may be similar in appearance, form, and function to the virtual indicators indicating positions of soft tissue attachment points but indicate portions of bone to remove on a non-viewable portion of the bone. In some examples, MR visualization device 104 may present virtual indicators indicating positions of bone to remove even in surgical procedures that do not involve detachment of soft tissue attachment points. In general, discussion in this disclosure MR visualization related to soft tissue attachment points is applicable to portions of bones.

The operation of FIG. 6 may be repeated multiple times during a surgery. For instance, after attempting to remove one or more soft tissue attachment points, clinician 110 may use ultrasound probe 106 again. Processing system 402 may therefore obtain second ultrasound imaging data representing the non-viewable portion of the bone at a later time than the first ultrasound data. Processing system 402 may determine, based on the second ultrasound imaging data, whether the soft tissue attachment point continues to exist. Based on the soft tissue attachment point not continuing to exist, processing system 402 may cause the MR visualization device 104 not to display the virtual indicator superimposed on the viewable portion of the bone. In some examples, processing system 402 may determine, based on the second ultrasound imaging data, that there are no remaining soft tissue attachment points at which soft tissue structures are attached to the bone. Based on there being no remaining soft tissue attachment points, processing system 402 may cause the MR visualization device 104 to a present a message that the bone may be removed from patient 112.

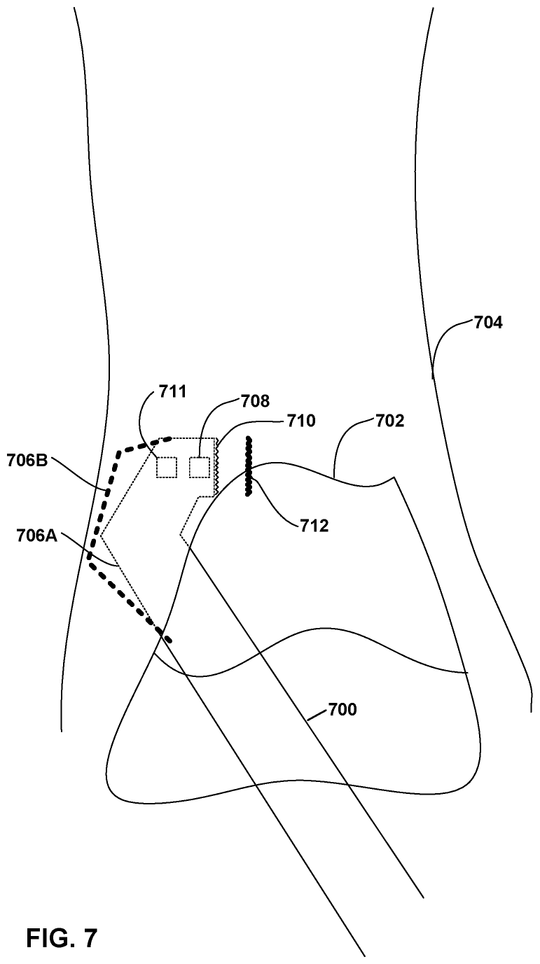

In some examples, processing system 402 may cause MR visualization device 104 to present instructions to use a detachment tool to detach the soft tissue structure from the bone. For instance, FIG. 7 is a conceptual diagram illustrating an example MR visualization in which virtual elements indicate locations of a detachment tool 700 and resection surface in accordance with one or more techniques of this disclosure. A resection surface may be a blade, burr, trimming tool, or other soft tissue resection device. In the example of FIG. 7, clinician 110 is using a detachment tool 700 inserted through an incision 702 in a leg 704 of a patient. Processing system 402 may cause MR visualization device 104 to present instructions to position a detachment tool 700 at an initial location. In the example of FIG. 7, instructions 706 are in the form of outlines 706A and 706B. Outline 706A represents a current position of detachment tool 700. Outline 706B represents a target position of detachment tool 700. In other examples, the instructions may have other forms, such as text, arrows, and so on.

Detachment tool 700 may comprise a motor 708. While detachment tool 700 is at the initial location, motor 708 may advance a resection surface 710 of detachment tool 700 to detach the soft tissue structure from the bone without manual movement of detachment tool 700. In the example of FIG. 7, resection surface 710 is a blade. In other examples, resection surface 710 may be a burr, trimming tool, or other soft tissue resection device. Furthermore, in some such examples, detachment tool 700 may comprise a sensor 711 that generates a signal based on resistance to advancement of resection surface 710. Detachment tool 700 may be configured to control advancement of resection surface 710 based on the resistance to the advancement of resection surface 710. For instance, there may be more resistance to the advancement of resection surface 710 when resection surface 710 is cutting through the soft tissue than when resection surface 710 has completed cutting the soft tissue. In this way, after clinician 110 has initially placed detachment tool 700, detachment tool 700 may automatically advance resection surface 710 to cut a soft tissue attachment point. In some examples, processing system 402 may cause MR visualization device 104 to present information related to a position of resection surface 710 after detachment tool 700 is at the initial location. For instance, MR visualization device 104 may display a virtual element 712 indicating a current location of resection surface 710 overlaid on the anatomy of patient 112. In some examples, MR visualization device 104 or one or more other devices may provide indications when detachment of a soft tissue attachment point complete or approaching completion.

In some examples where processing system 402 is implemented in computing system 102, processing system 402 may cause MR visualization device 104 to display the virtual indicator may sending instructions to MR visualization device 104 to display the virtual indicator. In other examples, such as examples where processing system 402 is implemented at least in part in MR visualization device 104, processing system 402 may directly cause output device(s) 510 of MR visualization device 104 to display the virtual indicator.

The following is a non-limiting set of examples that are in accordance with one or more techniques of this disclosure.
Clause 1. A method comprising: obtaining, by a processing system comprising one or more processors implemented in circuitry, ultrasound imaging data representing a non-viewable portion of a bone of a patient; identifying, by the processing system, based on the ultrasound imaging data, a soft tissue attachment point, wherein the soft tissue attachment point is a point on the non-viewable portion of the bone at which a soft tissue structure is attached to the bone; and causing, by the processing system, a Mixed Reality (MR) visualization device to display a virtual indicator superimposed on a viewable portion of the bone at a location corresponding to the soft tissue attachment point.
Clause 2. The method of clause 1, wherein: identifying the soft tissue attachment point comprises determining coordinates of the soft tissue attachment point in a virtual coordinate system of an ultrasound-based 3-dimensional (3D) model, and the method further comprises: obtaining a 3D reference bone model of the bone; generating first registration data that registers the virtual coordinate system of the ultrasound-based 3D model with a virtual coordinate system of the 3D reference bone model, wherein the ultrasound imaging data includes the ultrasound-based 3D model; generating second registration data that registers a coordinate system of the MR visualization device with the virtual coordinate system of the 3D reference bone model; and determining, based on the first registration data and the second registration data, a location in the coordinate system of the MR visualization device of the location corresponding to the soft tissue attachment point.
Clause 3. The method of any of clauses 1-2, wherein the soft tissue structure is one of: a tendon, a ligament, a muscle, cartilage, scar tissue, tough capsule tissue, or a blood vessel.
Clause 4. The method of any of clauses 1-3, wherein the non-viewable portion of the bone and the viewable portion of the bone are opposite sides of the bone.
Clause 5. The method of clause 4, wherein the bone is a tibia, the non-viewable portion of the bone is an area on a posterior side of the tibia, and the viewable portion of the bone is an area on an anterior side of the tibia at which an incision to access the tibia has been made.
Clause 6. The method of clause 4, wherein the non-viewable portion of the bone is an area on a first side of the bone, the viewable portion of the bone is an area on a second side of the bone opposite the first side of the bone at which an incision to access the bone has been made.
Clause 7. The method of any of clauses 1-6, wherein: the ultrasound imaging data is first ultrasound imaging data, and the method further comprises: obtaining second ultrasound imaging data representing the non-viewable portion of the bone at a later time than the first ultrasound imaging data; determining, by the processing system, based on the second ultrasound imaging data, whether the soft tissue attachment point continues to exist; and based on the soft tissue attachment point not continuing to exist, causing, by the processing system, the MR visualization device not to display the virtual indicator superimposed on the viewable portion of the bone.
Clause 8. The method of clause 7, further comprising: determining, by the processing system, based on the second ultrasound imaging data, that there are no remaining soft tissue attachment points at which soft tissue structures are attached to the bone; and based on there being no remaining soft tissue attachment points, causing, by the processing system, the MR visualization device to a present a message that the bone may be removed from the patient.
Clause 9. The method of any of clauses 1-8, further comprising causing, by the processing system, the MR visualization device to present instructions to use a detachment tool to detach the soft tissue structure from the bone.
Clause 10. The method of clause 9, wherein: causing the MR visualization device to present instructions to use the detachment tool comprise causing, by the processing system, the MR visualization device to present instructions to position the detachment tool at an initial location, and the detachment tool comprises a motor configured such that, while the detachment tool is at the initial location, advance a resection surface of the detachment tool to detach the soft tissue structure from the bone without manual movement of the detachment tool.
Clause 11. The method of clause 10, wherein: the detachment tool comprises a sensor that generates a signal based on resistance to advancement of the resection surface, and the detachment tool is configured to control advancement of the resection surface based on the resistance to the advancement of the resection surface.
Clause 12. The method of clause 10, further comprising causing, by the processing system, the MR visualization device to present information related to a position of the resection surface after the detachment tool is at the initial location.
Clause 13. The method of any of clauses 1-4 or 6-12, wherein the bone is one of: a femur, fibula, talus, vertebra, ilium, scapula, humerus, radius, or ulna.
Clause 14. The method of any of clauses 1-13, wherein identifying the soft tissue attachment point comprises applying, by the processing system, a machine learning model that identifies the soft tissue attachment point based on the ultrasound imaging data.
Clause 15. The method of any of clause 1-14, wherein the virtual indicator is a first virtual indicator and the method further comprises causing, by the processing system, the MR visualization device to display a second virtual indicator superimposed on the viewable portion of the bone at a location corresponding to target bone tissue on the non-viewable portion of the bone or another bone of the patient.
Clause 16. A computing system comprising: a storage system configured to store data depicting an ultrasound image representing a non-viewable portion of a bone of a patient; a processing system comprising one or more processors implemented in processing circuitry, the processing system configured to: obtain ultrasound imaging data representing the non-viewable portion of the bone of the patient; identify, based on the ultrasound imaging data, a soft tissue attachment point, wherein the soft tissue attachment point is a point on the non-viewable portion of the bone at which a soft tissue structure is attached to the bone; and cause, a Mixed Reality (MR) visualization device to display a virtual indicator superimposed on a viewable portion of the bone at a location corresponding to the soft tissue attachment point.
Clause 17. The computing system of clause 16, wherein the processing system is further configured to: determine coordinates of the soft tissue attachment point to identify the soft tissue attachment point; determine coordinates of the soft tissue attachment point in a virtual coordinate system of an ultrasound-based 3-dimensional (3D) bone model; obtain a 3D reference bone model of the bone; generate first registration data that registers the virtual coordinate system of the ultrasound-based 3D model with a virtual coordinate system of the 3D reference bone model, wherein the ultrasound imaging data includes the ultrasound-based 3D model; generate second registration data that registers a coordinate system of the MR visualization device with the virtual coordinate system of the 3D reference bone model; and determine, based on the first registration data and the second registration data, a location in the coordinate system of the MR visualization device of the location corresponding to the soft tissue attachment point.
Clause 18. The computing system of any of clauses 16-17, wherein the soft tissue structure is one of: a tendon, a ligament, a muscle, cartilage, scar tissue, tough capsule tissue, or a blood vessel.
Clause 19. The computing system of any of clauses 15-18, wherein the non-viewable portion of the bone and the viewable portion of the bone are opposite sides of the bone.
Clause 20. The computing system of clause 19, wherein the bone is a tibia, the non-viewable portion of the bone is an area on a posterior side of the tibia, the viewable portion of the bone is an area on an anterior side of the tibia at which an incision to access the tibia has been made.
Clause 21. The computing system of clause 19, wherein the non-viewable portion of the bone is an area on a first side of the bone, the viewable portion of the bone is an area on a second side of the bone opposite the first side of the bone at which an incision to access the bone has been made.
Clause 22. The computing system of any of clauses 16-21, wherein: the ultrasound imaging data is first ultrasound imaging data, and the processing system is further configured to: obtain second ultrasound imaging data representing the non-viewable portion of the bone at a later time than the first ultrasound imaging data; determine, based on the second ultrasound imaging data, whether the soft tissue attachment point continues to exist; and based on the soft tissue attachment point not continuing to exist, causing, the MR visualization device not to display the virtual indicator superimposed on the viewable portion of the bone.
Clause 23. The computing system of clause 22, wherein the processing system is further configured to: determine, based on the second ultrasound imaging data, that there are no remaining soft tissue attachment points at which soft tissue structures are attached to the bone; and based on there being no remaining soft tissue attachment points, causing, the MR visualization device to a present a message that the bone may be removed from the patient.
Clause 24. The computing system of any of clauses 16-23, wherein the processing system is further configured to cause the MR visualization device to present instructions to use a detachment tool to detach the soft tissue structure from the bone.
Clause 25. The computing system of any of clauses 16-19 or 21-24, wherein the bone is one of: a femur, fibula, talus, vertebra, ilium, scapula, humerus, radius, or ulna.
Clause 26. The computing system of any of clauses 16-25, wherein the processing system is configured to, as part of identifying the soft tissue attachment point, apply, a machine learning model that identifies the soft tissue attachment point based on the ultrasound imaging data.
Clause 27. A non-transitory computer-readable storage medium having instructions stored thereon that, when executed, cause a processing system to perform the methods of any of clauses 1-15.
Clause 28. A system comprising means for performing the methods of any of clauses 1-15.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, storage system, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein.

Various examples have been described. These and other examples are within the scope of the following claims.

1. A method comprising:
obtaining, by a processing system comprising one or more processors implemented in circuitry, ultrasound imaging data representing a non-viewable portion of a bone of a patient;
identifying, by the processing system, based on the ultrasound imaging data, a soft tissue attachment point, wherein the soft tissue attachment point is a point on the non-viewable portion of the bone at which a soft tissue structure is attached to the bone; and
causing, by the processing system, a Mixed Reality (MR) visualization device to display a virtual indicator superimposed on a viewable portion of the bone at a location corresponding to the soft tissue attachment point.

2. The method of claim 1, wherein:
identifying the soft tissue attachment point comprises determining coordinates of the soft tissue attachment point in a virtual coordinate system of an ultrasound-based 3-dimensional (3D) model, and
the method further comprises:
obtaining a 3D reference bone model of the bone;
generating first registration data that registers the virtual coordinate system of the ultrasound-based 3D model with a virtual coordinate system of the 3D reference bone model, wherein the ultrasound imaging data includes the ultrasound-based 3D model;
generating second registration data that registers a coordinate system of the MR visualization device with the virtual coordinate system of the 3D reference bone model; and
determining, based on the first registration data and the second registration data, a location in the coordinate system of the MR visualization device of the location corresponding to the soft tissue attachment point.

3. The method of claim 1, wherein the soft tissue structure is one of: a tendon, a ligament, a muscle, cartilage, scar tissue, tough capsule tissue, or a blood vessel.

4. The method of claim 1, wherein the non-viewable portion of the bone and the viewable portion of the bone are opposite sides of the bone.

5. The method of claim 4, wherein the bone is a tibia, the non-viewable portion of the bone is an area on a posterior side of the tibia, and the viewable portion of the bone is an area on an anterior side of the tibia at which an incision to access the tibia has been made.

6. The method of claim 4, wherein the non-viewable portion of the bone is an area on a first side of the bone, the viewable portion of the bone is an area on a second side of the bone opposite the first side of the bone at which an incision to access the bone has been made.

7. The method of claim 1, wherein:
the ultrasound imaging data is first ultrasound imaging data, and
the method further comprises:
obtaining, by the processing system, second ultrasound imaging data representing the non-viewable portion of the bone at a later time than the first ultrasound imaging data;
determining, by the processing system, based on the second ultrasound imaging data, whether the soft tissue attachment point continues to exist; and
based on the soft tissue attachment point not continuing to exist, causing, by the processing system, the MR visualization device not to display the virtual indicator superimposed on the viewable portion of the bone.

8. The method of claim 7, further comprising:
determining, by the processing system, based on the second ultrasound imaging data, that there are no remaining soft tissue attachment points at which soft tissue structures are attached to the bone; and
based on there being no remaining soft tissue attachment points, causing, by the processing system, the MR visualization device to a present a message that the bone may be removed from the patient.

9. (canceled)

10. The method of claim 1, further comprising:
causing, by the processing system, the MR visualization device to present instructions to position a detachment tool at an initial location, and
the detachment tool comprises a motor configured such that, while the detachment tool is at the initial location, advance a resection surface of the detachment tool to detach the soft tissue structure from the bone without manual movement of the detachment tool.

11. The method of claim 10, wherein:
the detachment tool comprises a sensor that generates a signal based on resistance to advancement of the resection surface, and
the detachment tool is configured to control advancement of the resection surface based on the resistance to the advancement of the resection surface.

12. The method of claim 10, further comprising causing, by the processing system, the MR visualization device to present information related to a position of the resection surface after the detachment tool is at the initial location.

13. (canceled)

14. The method of claim 1, wherein identifying the soft tissue attachment point comprises applying, by the processing system, a machine learning model that identifies the soft tissue attachment point based on the ultrasound imaging data.

15. The method of claim 1, wherein the virtual indicator is a first virtual indicator and the method further comprises causing, by the processing system, the MR visualization device to display a second virtual indicator superimposed on the viewable portion of the bone at a location corresponding to target bone tissue on the non-viewable portion of the bone or another bone of the patient.

16. A computing system comprising:
a storage system configured to store ultrasound imaging data representing a non-viewable portion of a bone of a patient; and
a processing system comprising one or more processors implemented in processing circuitry, the processing system configured to:
identify, based on the ultrasound imaging data, a soft tissue attachment point, wherein the soft tissue attachment point is a point on the non-viewable portion of the bone at which a soft tissue structure is attached to the bone; and
cause, a Mixed Reality (MR) visualization device to display a virtual indicator superimposed on a viewable portion of the bone at a location corresponding to the soft tissue attachment point.

17. The computing system of claim 16, wherein the processing system is further configured to:
determine coordinates of the soft tissue attachment point to identify the soft tissue attachment point;
determine coordinates of the soft tissue attachment point in a virtual coordinate system of an ultrasound-based 3-dimensional (3D) bone model;
obtain a 3D reference bone model of the bone;
generate first registration data that registers the virtual coordinate system of the ultrasound-based 3D model with a virtual coordinate system of the 3D reference bone model, wherein the ultrasound imaging data includes the ultrasound-based 3D model;
generate second registration data that registers a coordinate system of the MR visualization device with the virtual coordinate system of the 3D reference bone model; and
determine, based on the first registration data and the second registration data, a location in the coordinate system of the MR visualization device of the location corresponding to the soft tissue attachment point.

18. (canceled)

19. The computing system of claim 16, wherein the non-viewable portion of the bone and the viewable portion of the bone are opposite sides of the bone.

20. (canceled)

21. The computing system of claim 19, wherein the non-viewable portion of the bone is an area on a first side of the bone, the viewable portion of the bone is an area on a second side of the bone opposite the first side of the bone at which an incision to access the bone has been made.

22. The computing system of claim 16, wherein:
the ultrasound imaging data is first ultrasound imaging data, and
the processing system is further configured to:
obtain second ultrasound imaging data representing the non-viewable portion of the bone at a later time than the first ultrasound imaging data;
determine, based on the second ultrasound imaging data, whether the soft tissue attachment point continues to exist; and
based on the soft tissue attachment point not continuing to exist, causing, the MR visualization device not to display the virtual indicator superimposed on the viewable portion of the bone.

23. The computing system of claim 22, wherein the processing system is further configured to:
determine, based on the second ultrasound imaging data, that there are no remaining soft tissue attachment points at which soft tissue structures are attached to the bone; and
based on there being no remaining soft tissue attachment points, causing, the MR visualization device to a present a message that the bone may be removed from the patient.

24-26. (canceled)

27. One or more non-transitory computer-readable storage media having instructions stored thereon that, when executed, cause a processing system to:
obtain ultrasound imaging data representing a non-viewable portion of a bone of a patient;
identify, based on the ultrasound imaging data, a soft tissue attachment point, wherein the soft tissue attachment point is a point on the non-viewable portion of the bone at which a soft tissue structure is attached to the bone; and
cause a Mixed Reality (MR) visualization device to display a virtual indicator superimposed on a viewable portion of the bone at a location corresponding to the soft tissue attachment point.

28. (canceled)
